(19) United States
(12) Patent Application Publication
(10) Pub. No.: US 2004/0072223 A1
(43) Pub. Date: Apr. 15, 2004

(54) Method for detecting macromolecular biopolymers by using at least one immobilization unit provided with a marked scavenger molecule

(76) Inventors: R. Johannes Luyken, Munchen (DE); Franz Hofmann, Munchen (DE)

Correspondence Address: Jeffrey R Stone, Briggs & Morgan, 2400 IDS Center, Minneapolis, MN, 55402, US

(21) Appl. No.: 10/469,274

(22) PCT Filed: Mar. 1, 2002
(86) PCT No.: PCT/DE02/00760

(30) Foreign Application Priority Data
Mar. 1, 2001 (DE) 10109779.4

(51) Int. Cl. C12Q 1/68; G01N 33/53; G01N 33/537; G01N 33/543

(52) U.S. Cl. 435/6; 435/7.92

(57) ABSTRACT

A device and method for detecting macromolecular biopolymers by using at least one unit for immobilizing biopolymers. In the device, the unit for immobilizing macromolecular biopolymers is provided with scavenger molecules that may bind macromolecular biopolymers and that have a label capable of generating a detectable signal. A detection unit in the device uses the label to detect macromolecular biopolymers bound to the scavenger molecules. The method is based on the knowledge that the macromolecular biopolymers to be detected are not provided with a label but that the scavenger molecules are provided with a label prior to immobilization

The invention relates to a device and a method for detecting macromolecular biopolymers by using at least one unit for immobilizing macromolecular biopolymers.

[1] to [4] disclose methods for detecting DNA molecules, in which biosensors based on electrode arrangements are used for the detection.

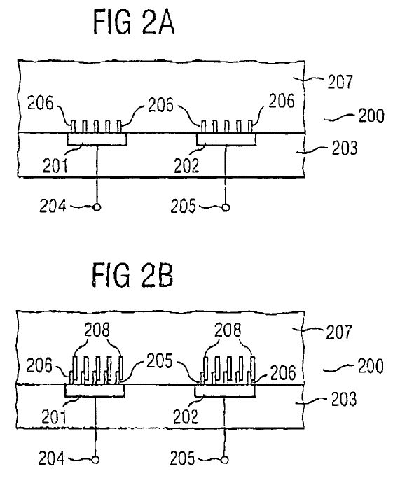

FIG. 2a and FIG. 2b depict a sensor of the kind described in [1] and [4]. The sensor 200 has two electrodes 201, 202 made of gold, which are embedded in an insulator layer 203 made of insulator material. Electrode terminals 204, 205, to which the electrical potential applied to the electrode 201, 202 can be delivered, are connected to the electrodes 201, 202. The electrodes 201, 202 are arranged as planar electrodes. DNA probe molecules 206 are immobilized on each electrode 201, 202 (cf. FIG. 2a). The immobilization is carried out according to the so-called gold-sulfur coupling. The analyte to be tested, for example an electrolyte 207, is applied to the electrodes 201, 202.

If the electrolyte 207 contains DNA strands 208 with a sequence which is complementary to the sequence of the DNA probe molecules 206, then these DNA strands 208 hybridize with the DNA probe molecules 206 (cf. FIG. 2b).

Hybridization of a DNA probe molecule 206 and a DNA strand 208 takes place only if the sequences of the particular DNA probe molecule 206 and the corresponding DNA strand 208 are complementary to one another. If this is not the case, then no hybridization takes place. A DNA probe molecule with a predetermined sequence is thus in each case only capable of binding, i.e. hybridizing, to a particular DNA strand, namely the one with the respective complementary sequence.

If hybridization takes place, the capacitance between the electrodes is altered, as can be seen from FIG. 2b. This alteration in capacitance is used as measured variable for detecting DNA molecules.

[5] discloses another procedure for studying the electrolyte for the existence of a DNA strand with predetermined sequence. In this procedure, the DNA strands of the desired sequence are labeled with a fluorescent dye and their existence is determined on the basis of the reflection properties of the labeled molecules. For this purpose, the electrolyte is illuminated with light in the visible wavelength range and the light reflected by the electrolyte, in particular by the labeled DNA strand to be detected, is detected. Owing to the reflection behavior, i.e. in particular owing to the reflected light beams detected, it is determined, whether or not the DNA strand with the correspondingly predetermined sequence, which is to be detected, is present in the electrolyte.

This procedure is very complicated, since it demands a very exact knowledge about the reflection behavior of the corresponding DNA strand and furthermore requires labeling of the DNA strands prior to the process. Furthermore, the means of detecting the reflected light beams needs to be adjusted very accurately, in order to be able to detect the reflected light beams at all.

Thus, said procedure is expensive, complicated and very sensitive to disturbing influences, and, as a result, it is very readily possible for the measurement result to be distorted.

It is furthermore known from affinity chromatography (cf. [6]) to use immobilized low molecular weight molecules, in particular ligands of high specificity and affinity, in order to specifically bind peptides and proteins, e.g. enzymes, in the analyte.

It is furthermore also known that in detection methods for antigens or antibodies, such as the “ELISA” tests, which are based on solid phase systems, one of the two reaction partners is bound to a solid phase (e.g. microtiter plates). After the antibody-antigen reaction has taken place, it is detected by a labeled reaction partner (cf. [7]). More precisely, such an antibody capture assay comprises firstly binding the antigen to a solid support. Secondly, a labeled antibody present in a solution reacts with the antigen. After washing off the unbound antibody, a qualitative or quantitative answer is obtained by measuring the label on the bound antibody (cf. [8] and [9]).

[2] and [3] furthermore disclose a reduction/oxidation recycling method for detecting macromolecular biopolymers.

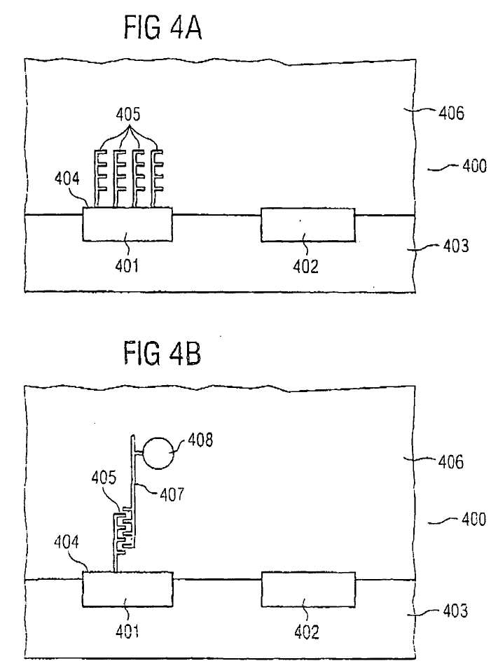

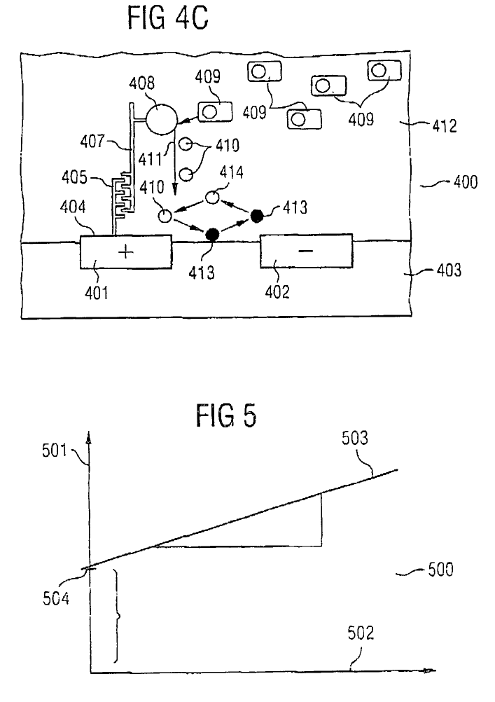

The reduction/oxidation recycling method, also referred to as redox recycling method hereinbelow, will be illustrated in more detail on the basis of FIG. 4a to FIG. 4c hereinbelow.

FIG. 4a depicts a biosensor 400 having a first electrode 401 and a second electrode 402 which are applied to a substrate 403 as insulator layer.

A holding region, configured as holding layer 404, is applied to the first electrode 401 made of gold. The holding region serves to immobilize DNA probe molecules 405 on the first electrode 401.

There is no such holding region provided on the second electrode.

If DNA strands with a sequence which is complementary to the sequence of the DNA probe molecules 405 are to be detected by means of the biosensor 400, the sensor 400 is contacted with a solution 406 to be studied, for example an electrolyte, in such a manner that any DNA strands which may be present in the solution 406 to be studied and which have the sequence complementary to the sequence of the DNA probe molecules 405 can hybridize.

FIG. 4b depicts the case in which the solution 406 to be studied contains the DNA strands 407 to be detected which have hybridized to the DNA probe molecules 405.

The DNA strands 407 in the solution to be studied are labeled with an enzyme 408 which makes it possible to cleave molecules described below into part molecules.

The number of DNA probe molecules 405 provided is usually considerably larger than the number of DNA strands 407 to be determined, which are present in the solution 406 to be studied.

After the DNA strands 407 which may be present in the solution 406 to be studied and which have the enzyme 408 have hybridized with the immobilized DNA probe molecules, the biosensor 400 is rinsed, thereby removing the unhybridized DNA strands and cleaning the biosensor 400 of the solution 406 to be studied.

An electrically uncharged substance which contains molecules which can be cleaved by the enzyme on the hybridized DNA strands 407 into a first part molecule of a negative first electrical charge and into a second part molecule of a positive second electrical charge is added to said rinsing solution used for rinsing or to a further solution 412 which is supplied specifically for this purpose in a further phase.

As shown in FIG. 4c, the negatively charged part molecules are attracted to the positively charged anode, as indicated by the arrow 411 in FIG. 4c.

The negatively charged first part molecules 410 are oxidized at the first electrode 401 which, as anode, has a positive electrical potential and are, as oxidized part molecules 413, attracted to the negatively charged cathode, i.e. the second electrode 402, where they are reduced again.

The reduced part molecules 414, in turn, migrate to the first electrode 401, i.e. to the anode.

In this way, an electrical cycle current is generated which is proportional to the number of charge carriers generated in each case by the enzymes 408.

The electrical parameter which is evaluated in this method is the change in the electric current

$$\frac{ⅆ I}{ⅆ t}$$

as a function of time t, as depicted in diagram 500 in FIG. 5.

The abovementioned methods for detecting macromolecular biopolymers have in common that the macromolecular biopolymers to be detected are labeled prior to carrying out the actual detection method. This is not only complicated and, for example, associated with the risk of possibly losing, for example, part of the sample to be studied or of the labeling process not being quantitative, but may also have other disadvantages. Thus, for example in the case of DNA molecules labeled with fluorescent dyes, the fluorescent labels may reduce the mobility of the DNA molecules and thus slow down the detection process.

[15] furthermore discloses a method for screening target-ligand interactions by using a chemical library of ligands, which method comprises measuring at least one fluorescence property of a chemical library of ligands immobilized on a solid phase, with a molecular fluorescence sensor being bound to each ligand, prior to and after addition of the target.

Furthermore, [16] discloses an in situ hybridization method in which transcription products of proteins of the bone matrix were detected in mouse tissue cells.

[17], in addition, discloses a self-addressable microelectronic device which is designed in such a way that it can actively conduct molecular biological multi-step reactions and multiplex reactions in microscopic formats.

[18] finally discloses a detection system which may be used, for example, in biochemical or pharmaceutical research and which has at least one immobilized binding component A with an at least one binding site for a detection species B and at least one detection species B which can bind to the binding component A.

It is the object of the present invention to provide an alternative method and a device for detecting macromolecular biopolymers.

The problem is achieved by the method and the device, which have the features according to the independent patent claims.

Said method for recording macromolecular biopolymers uses at least one unit for immobilizing macromolecular biopolymers.

In this connection, the at least one unit for immobilizing macromolecular biopolymers is (first) provided with scavenger molecules which firstly may bind macromolecular biopolymers and secondly have a label which can generate a detectable signal. In said method, a sample to be studied is then contacted with the at least one unit for immobilizing macromolecular biopolymers, it being possible for said sample to be studied to contain the macromolecular biopolymers to be detected. This is followed by macromolecular biopolymers present in the sample to be studied binding to the scavenger molecules. Subsequently, scavenger molecules to which no macromolecular biopolymers to be detected have bound are removed and the macromolecular biopolymers are detected by using the label.

In simple terms, the method of the present invention is based on the knowledge that the macromolecular biopolymers to be detected are not, as previously, provided with a label but that the scavenger molecules are provided with a label prior to immobilization. This has the advantage that the sample to be studied need no longer be subjected to a labeling reaction during which part of the sample or possibly the entire sample may be lost or labeling is not completed.

The device for detecting macromolecular biopolymers, which is disclosed herein, has at least one unit for immobilizing macromolecular biopolymers and a detection unit. In the device the at least one unit for immobilizing macromolecular biopolymers is provided with scavenger molecules which may bind macromolecular biopolymers and which have a label capable of generating a detectable signal. The detection unit in the device is configured in such a way that it detects by means of the label macromolecular biopolymers which have bound to the scavenger molecules.

In one embodiment, the device has multiple units for immobilizing macromolecular biopolymers in a regular arrangement (an array). In the device the at least one unit for immobilizing or the regular arrangement of said units is preferably applied to a CMOS camera or to a CCD.

In the method described herein the label generates a signal. In one configuration, such a signal is an electric current. In another configuration, the signal is visible light or UV light. The signal may also consist of radioactive radiation or X radiation.

It is apparent from this that it is possible to use in the method various types of label which is also referred to as reporter group hereinbelow.

Firstly, the label may be a (chemical) compound or group which is directly capable of generating a signal which can be used for detecting the macromolecular biopolymers. Generation of said signal may be induced externally, but it is also possible for the label to emit the signal without external stimulation. In the first case, the label is, for example, a fluorescent dye (fluorophore) or a chemiluminescent dye and, in the second case, it is a radioisotope, for example.

Secondly, the label may be a substance which generates only indirectly a signal for recording the macromolecular biopolymers, i.e. a substance which causes the generation of the signal. Such a reporter group may be, for example, an enzyme which catalyzes a chemical reaction which is then used for detecting the biopolymers. Examples of such enzymes are alkaline phosphatase, glutathione S-transferase, superoxide dismutase, horseradish peroxidase, alpha-galactosidase and beta-galactosidase. These enzymes are capable of cleaving suitable substrates which give colored end products or, for example, compounds which may be used in the reduction/oxidation recycling method described above. The group of labels which generate only indirectly a signal which can be used for detecting macromolecular biopolymers includes furthermore ligands for binding proteins and substrates for enzymes. Said labels are generally referred to herein as enzyme ligands. Examples of such enzyme ligands which may be used as labels are biotin, digoxigenin and substrates for the enzymes mentioned above.

Detecting, in accordance with the invention, means both qualitative and quantitative detection of macromolecular biopolymers in an analyte to be studied. This means that the term “detecting” also includes determining the absence of macromolecular biopolymers in the analyte.

“Unit for immobilization”, in accordance with the invention, means an arrangement which has a surface on which the scavenger molecules can be immobilized, i.e. to which the scavenger molecules can bind by means of physical or chemical interactions. These interactions include hydrophobic or ionic (electrostatic) interactions and covalent bonds. Examples of suitable surface materials which may be used for the at least one unit for immobilization are metals such as gold or silver, plastics such as polyethylene or polypropylene and inorganic substances such as silicon dioxide, for example in the form of glass.

An example of a physical interaction which causes immobilization of the scavenger molecules is adsorption to the surface. This type of immobilization may take place, for example, if the means for immobilization is a plastic material which is used for the production of microtiter plates (e.g. polypropylene). However, preference is given to the scavenger molecules being covalently linked to the unit for immobilizing, since this makes it possible to control the orientation of the scavenger molecules. The covalent linkage may be effected via any suitable linker chemistry.

In one embodiment of the method, the at least one unit for immobilizing is applied to an electrode or a photodiode.

In another embodiment, the at least one unit for immobilizing macromolecular biopolymers is a nanoparticle.

A nanoparticle, in accordance with the invention, means a particle which can be obtained by “nanostructure-generating methods”. Nanostructure-generating methods which may be used for generating such nanoparticles on suitable substrates are, for example, the use described in [12] and [13] of block copolymer microemulsions and the use described in [14] of colloidal particles as structurization masks. The method described in [14] is, in principle, similar to a lithographic method commonly used in the area of substrate structurization. It should therefore be emphasized here that a nanoparticle in accordance with the invention is consequently not limited to those particles which are obtained by any of the methods mentioned herein by way of example. Rather such a nanoparticle is any particle whose diameter is in the nanometer range, i.e. usually in the range from 2 to 50 nm, preferably in the range from 5 to 20 nm, particularly preferably in the range from 5 to 10 nm.

Consequently, a “unit for immobilization, which is a nanoparticle”, also referred to as nanoparticle-shape unit hereinbelow, is an above-described nanoparticle which has a surface on which the scavenger molecules can be immobilized, i.e. the nature of the surface is such that the scavenger molecules can bind to it by means of physical or chemical interactions. These interactions include hydrophobic or ionic (electrostatic) interactions and covalent bonds. Examples of suitable surface materials which may be used for the at least one nanoparticle-like unit for immobilization are metals such as gold or silver, semiconducting materials such as silicon, plastics such as polyethylene or polypropylene or silicon dioxide, for example in the form of glass. Nanoparticle-like units made of plastics and silicon dioxide are obtainable here by using the colloidal mask methods described in [14]. Nanoparticle-like units made of semiconducting materials such as silicon may, for example, also be produced by the Stranski-Kranstanov method. It is furthermore possible to obtain nanoparticle-like units made of silicon dioxide by oxidation of such nanoparticles made of silicon.

Owing to the above-described preparation methods, nanoparticle-like units for immobilizing, which are applied to suitable substrate surfaces (holding regions), for example of photodiodes or electrodes, are arranged in a regular way, with distances between one another in the range of some 10 nanometers, for example from approx. 10 to 30 nm, on said surfaces. The type of arrangement and the distance between the nanoparticles, as well as the size of the nanoparticles, depend on the particular method for forming said nanoparticles.

One advantage when using nanoparticle-shape units for immobilizing is the possibility of immobilizing on said nanoparticles a precisely defined number of scavenger molecules. This is particularly advantageous for quantitative detection of macromolecular biopolymers by means of the present method. Another advantage when using nanoparticles as units for immobilizing is provided by the fact that the distance between the nanoparticles, i.e. the spatial separation of the scavenger molecules, provides better spatial accessibility of said scavenger molecules to the macromolecular biopolymers binding thereto and thus increases the probability of an interaction. Moreover, the nanoparticle design increases the effective surface area.

Macromolecular biopolymers here mean nucleic acids such as DNA and RNA molecules or else shorter nucleic acids such as oligonucleotides with from 10 to 20 base pairs (bp). The nucleic acids may be double-stranded or else may have at least single-stranded regions or may be present as single strands, for example due to prior thermal denaturation (strand separation) for their detection. In this connection, the sequence of the nucleic acids to be detected may be predetermined, i.e. known, at least partially or completely. Other macromolecular biopolymers are proteins or peptides. These may be made up from the 20 amino acids normally found in proteins, but may also contain not naturally occurring amino acids or may be modified, for example by sugar residues (oligosaccharides), or contain post translation modifications. Furthermore, it is also possible to detect complexes of several different macromolecular biopolymers, for example complexes of nucleic acids and proteins.

If the macromolecular biopolymers to be detected are proteins or peptides, the preferred scavenger molecules used are ligands which can specifically bind the proteins or peptides to be detected. The scavenger molecules/ligands are preferably linked to the means for immobilization by covalent bonds.

Suitable ligands for proteins and peptides are low molecular weight enzyme agonists or enzyme antagonists, pharmaceuticals, sugars or antibodies or any molecule capable of specifically binding proteins or peptides.

If DNA molecules (nucleic acids or oligonucleotides) of a predetermined nucleotide sequence are detected by the method described herein, they are preferably detected in single-stranded form, i.e. they are, where appropriate, converted to single strands by denaturation, as illustrated above, prior to detection. In this case, the scavenger molecules used are then preferably DNA probe molecules having a sequence complementary to the single-stranded region. The DNA probe molecules, in turn, may have oligonucleotides or else longer nucleotide sequences, as long as the latter do not form any of the intermolecular structures which prevent hybridization of the probe molecule with the nucleic acid to be protected. However, it is also possible to use DNA-binding proteins or agents as scavenger molecule.

It should be noted that it is of course possible to detect by the present method not only a single species of biopolymers in a single set of measurements. Rather it is possible to detect a plurality of macromolecular biopolymers simultaneously or else successively. For this purpose, several types of scavenger molecules, each of which has a (specific) binding affinity for a particular biopolymer to be detected, may be bound on the units and/or several units for immobilizing may be used, each of said units for immobilizing only one type of scavenger molecule having bound to it. In these multiple determinations, a label which is distinguishable from the other labels is preferably used for each macromolecular biopolymer to be detected. It is, for example, possible to use two or more fluorophores as labels, each of said fluorophores preferably having a specific excitation and emission wavelength.

In a first method step, the at least one unit for immobilizing is provided with the scavenger molecules which have a label capable of generating a detectable signal.

A sample to be studied, preferably a liquid medium such as an electrolyte, is then contacted with the unit for immobilizing. This is carried out in such a way that the macromolecular biopolymers can bind to the scavenger molecules. In the case, that the medium contains a plurality of macromolecular biopolymers to be detected, the conditions are chosen in such a way that said biopolymers can bind in each case simultaneously or successively to their corresponding scavenger molecule.

After waiting for an appropriate period of time in order for the macromolecular biopolymers to be able to bind to the corresponding scavenger molecule or corresponding scavenger molecules, unbound scavenger molecules are removed from the unit or the units for immobilizing on which they are located.

If the macromolecular biopolymers detected are proteins or peptides, the unbound ligands used as scavenger molecules are removed from the at least one unit for immobilizing by contacting a material with the at least one unit for immobilizing, said material being capable of hydrolyzing the chemical bond between the ligand and the unit for immobilizing.

In the case of the scavenger molecules being low molecular weight ligands, the latter can, if unbound, also be removed enzymatically.

To this end, the ligands are covalently linked to the unit for immobilization via an enzymatically cleavable linkage, for example via an ester linkage.

In this case it is possible to use, for example, a carboxylic ester hydrolase (esterase) in order to remove unbound ligand molecules. This enzyme hydrolyzes the particular ester bond between the unit for immobilization and the particular ligand molecule which has not been bound by a peptide or protein. In contrast, the ester linkages between the unit for immobilizing and those molecules which have performed a binding interaction with peptides or proteins remain intact, due to the reduced steric accessibility resulting from the spatial occupation of the bound peptide or protein.

In the case of the scavenger molecules being DNA strands, the unbound probe molecules are removed enzymatically, for example with the aid of an enzyme having nuclease activity. The enzyme having nuclease activity used is preferably an enzyme which selectively breaks down single-stranded DNA. In this connection, the selectivity of the degrading enzyme for single-stranded DNA must be taken into account. If the enzyme selected for breaking down unhybridized DNA single strands does not have said selectivity, then the DNA to be detected which is present in the form of a double-stranded hybrid with the probe molecule may possibly and undesirably also likewise be broken down.

It is in particular possible to use DNA nucleases, for example a nuclease from mung beans, the nuclease P1 or the nuclease S1 for removing the unbound DNA probe molecules from the respective electrode. Likewise it is possible to use DNA polymerases which due to their 5′→3′ exonuclease activity or their 3′→5′ exonuclease activity, are capable of breaking down single-stranded DNA.

After removing the unbound scavenger molecules, the macromolecular biopolymers are detected using the label. For this purpose, either a signal emitted by the label spontaneously, such as radioactive radiation, or by a signal caused by external stimulation, such as emitted fluorescence radiation, is measured.

If the signal measured is emitted fluorescence radiation, the biosensor may be designed in such a way that the measurement is carried out in a space-resolved manner directly on the sensor by applying, for example, the unit for immobilization directly to a photocell used for measurement and connecting said photocell to a corresponding evaluation unit. The advantage of this is a simplified measuring arrangement. Such a measuring arrangement can be made, for example, using a conventional CMOS camera or a CCD. However, it is of course also possible to use an external unit for detecting the emitted fluorescence radiation.

Depending on the label used and the method of measurement, it is also possible to carry out a measurement of the signal prior to or after providing the at least one unit for immobilizing macromolecular biopolymers with the scavenger molecules. In this case, the values determined from the two measurements of the signal are compared with one another. If the signal intensity of the measured values differ in such a way that the difference between the values determined is greater than a predetermined threshold value, it is assumed that macromolecular biopolymers have bound to scavenger molecules and thereby caused the intensity of the signal received by the receiver to change.

Exemplary embodiments of the invention, which will be illustrated in more detail below are depicted in the figures in which

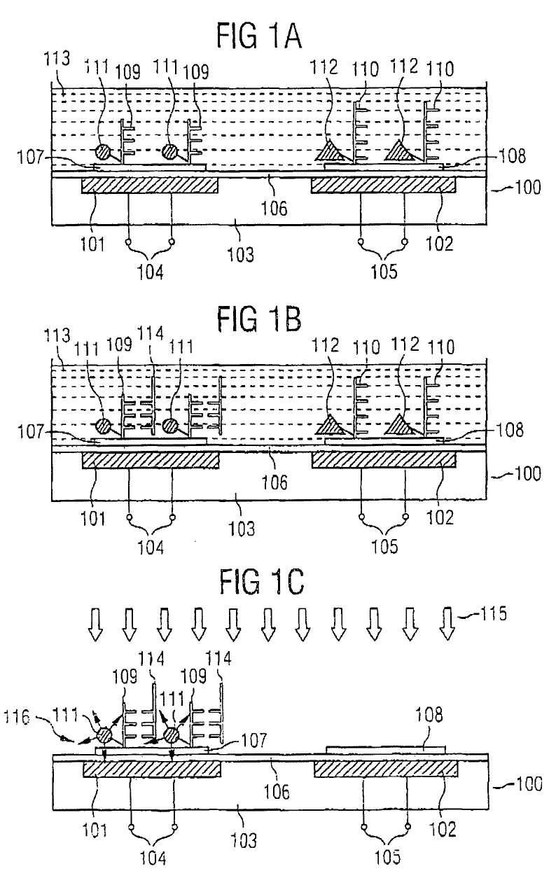

FIGS. 1a to 1c show a biosensor at different method stages on the basis of which the method according to an exemplary embodiment of the invention is illustrated;

FIGS. 2a and 2b show a sketch of two planar electrodes which can be used to detect the existence of DNA strands to be detected in an electrolyte (FIG. 2a) or the nonexistence thereof (FIG. 2b);

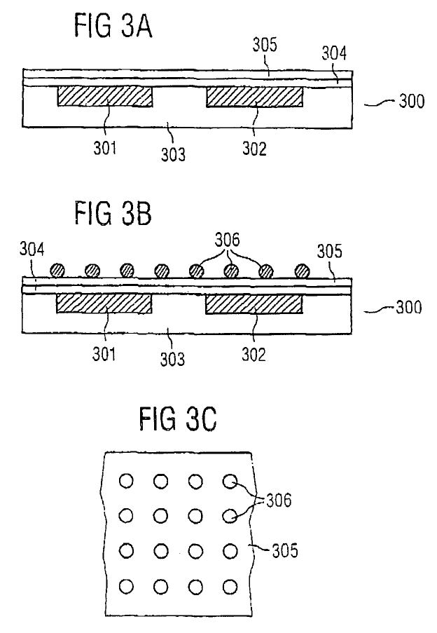

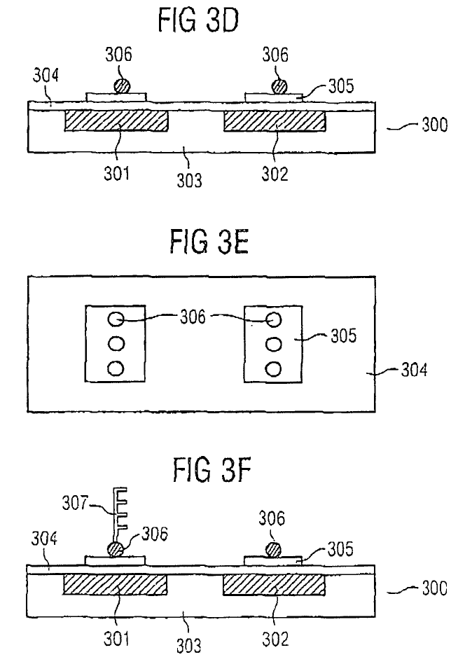

FIGS. 3a to 3f show a biosensor which can be used to carry out another embodiment of the method described herein;

FIGS. 4a to 4c show sketches of a biosensor according to the prior art, on the basis of which individual states as part of the redox recycling process are explained;

FIG. 5 shows a functional curve of a circuit current in accordance with the prior art as part of a redox recycling process;

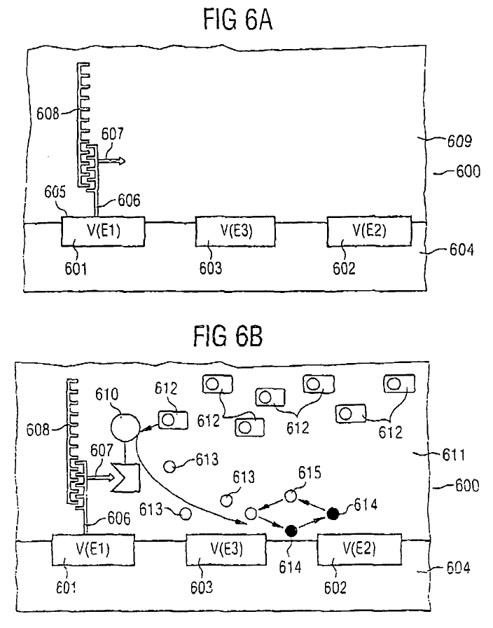

FIGS. 6a and 6b show a biosensor which can be used to carry out a redox recycling process as further embodiment of the method.

FIG. 1 shows a section from a biosensor 100 which can be used to carry out a first exemplary embodiment of the method described herein.

FIG. 1a depicts the biosensor 100 having a first photodiode 101 and a second photodiode 102 which are arranged in an insulator layer 103 made of insulator material.

The first photodiode 101 and the second photodiode 102 are connected via first electrical terminals 104 and, respectively, second electrical terminals 105 to an evaluation unit (not shown). The two photodiodes 101, 102 are furthermore provided with an oxide layer 106 and a first unit 107 for immobilizing macromolecular biopolymers and, respectively a second unit 108 for immobilizing macromolecular biopolymers. The units for immobilizing, 107 and 108, are prepared from gold.

Alternatively, the units 107, 108 for immobilizing may also be prepared from silicon oxide and coated with a material which is suitable for immobilizing scavenger molecules.

It is possible, for example, to use known alkoxy silane derivatives such as

3-glycidoxypropylmethoxysilane,

3-acetoxypropyltrimethoxysilane,

3-aminopropyltriethoxysilane,

4-(hydroxybutyramido)propyltriethyoxysilane,

3-N,N-bis(2-hydroxyethyl)aminopropyltriethoxysilane, or other related materials which are capable of forming, with their one end, a covalent bond with the surface of the silicon oxide and, with their other end, providing the probe molecule to be immobilized with a chemically reactive group such as an epoxy, acetoxy, amine or hydroxyl radical for reaction.

If a scavenger molecule to be immobilized reacts with an activated group of this kind, then it will be bound via the chosen material as a kind of covalent linker to the surface of the coating on the unit for immobilizing.

DNA probe molecules 109, 110 are applied as scavenger molecules to the units for immobilizing 107 and 108.

In this connection, first DNA probe molecules 109 having a sequence complementary to a predetermined first DNA sequence are applied to the first photodiode 101 by means of the unit 107. The DNA probe molecules 109 are in each case labeled with a first fluorophore 111.

The fluorophore used may be, for example, fluorescein. The scavenger molecules 109 may be labeled by incorporating an appropriately labeled nucleotide such as ChromaTide Fluorescein-12-dUTP (Molecular Probes, Inc., Eugene, Oreg., USA, Product No. C-7604) enzymatically, i.e. by means of suitable polymerases such as DNA polymerase or Klenow polymerase, into the oligonucleotides (scavenger molecules) 109 (cf. [10]).

Second DNA probe molecules 110 having a sequence which is complementary to a predetermined second DNA sequence are applied to the second photodiode 102. The DNA probe molecules 110 are in each case labeled with a second fluorophore 112. The label 112 used may be, for example, the fluorophore “Oregon Green™ 488” which is, likewise coupled to a nucleotide such as dUTP (Molecular Probes, Inc., Eugene, Oreg., USA, Product No. C7630), enzymatically incorporated into the DNA molecules 110.

Sequences of DNA strands, which are in each case complementary to the sequences of the probe molecules can hybridize in the usual manner to the pyrimidine bases adenine (A), guanine (G), thymine (T) or uracil (U) in the case of an above-described label, or cytosine (C), i.e. by base pairing via hydrogen bridge bonds between A and T or U and between C and G.

FIG. 1a furthermore depicts an electrolyte 113 which is contacted with the photodiodes 101, 102 and the DNA probe molecules 108, 109.

FIG. 1b depicts the biosensor 100 in the case that the electrolyte 113 contains DNA strands 114 which have a predetermined first nucleotide sequence which is complementary to the sequence of the first DNA probe molecules 109.

In this case, the DNA strands 114 complementary to the first DNA probe molecules 109 hybridize with said first DNA probe molecules 109 which have been applied to the first photodiode 101.

Since the sequences of DNA strands hybridize only with the in each case specific complementary sequence, the DNA strands complementary to the first DNA probe molecules do not hybridize with the second DNA probe molecules 110.

As can be seen from FIG. 1b, the result after hybridization has been carried out is that hybridized molecules are located, i.e. double-stranded DNA molecules are immobilized, on the first photodiode 101. Only the second DNA probe molecules 110 as still single-stranded molecules are present on the second photodiode 102.

In a further step, hydrolysis of the single-stranded DNA probe molecules 110 on the second photodiode 102 is effected by means of a biochemical method, for example by adding DNA nucleases to the electrolyte 113.

Here, the selectivity of the degrading enzyme for single-stranded DNA must be taken into account. If the enzyme selected for breaking down the non-hybridized DNA single strands does not have this selectivity, then the nucleic acid to be detected, which is present as double-stranded DNA, is possibly likewise (undesirably) broken down, which would cause a distortion of the result of the measurement.

After removing the single-stranded DNA probe molecules, i.e. the second DNA probe molecules 110 on the second photodiode 102, only the hybrids of the DNA molecules 114 to be detected and the first DNA probe molecules 109 complementary thereto (cf. FIG. 1c) are present.

In order to remove the unbound single-stranded DNA probe molecules 110 on the second photodiode 102, i.e. the second unit for immobilizing, one of the following substances may be added, for example:

nuclease from mung beans,

nuclease P1 or

nuclease S1.

For this purpose, it is likewise possible to use DNA polymerases which are capable of breaking down single-stranded DNA, owing to their 5′→3′ exonuclease activity or their 3′→5′ exonuclease activity.

After degradation of the single-strand probe molecules, the electrolyte may, where appropriate, be removed from the photodiodes 101 and 102. This increases the contrast, i.e. reduces the background, for the subsequent fluorescence measurement.

A laser, not shown, is then used to irradiate with light which is symbolized by arrows 115 and which has a wavelength suitable for making the first fluorophore 111 and the second fluorophore 112 fluoresce. Depending on the type of fluorophores, it is also possible to use different wavelengths, either simultaneously or else successively.

The irradiated light makes only the fluorophore 111 which is located on the first DNA probe molecules 109 emit, since the unbound second DNA probe molecules 110 including the fluorophore 112 have been removed from the second photodiode 102 by the nuclease treatment (cf. FIG. 1c). The fluorescence radiation symbolized by the arrow 116, which is emitted by the fluorophore 111 is detected by the first photodiode 101. The second photodiode 102, however, does not detect any fluorescence radiation.

In this way the presence of the DNA molecules 114 is determined. The use of the biosensor 100 described herein permits a spatially resolved detection and offers a distinct simplification of the entire measuring arrangement, since there is no need for an external unit for detecting the fluorescence radiation.

FIG. 3 depicts a section of a biosensor 300 which is configured with at least one unit for immobilization in the form of nanoparticles and which can be used to carry out another embodiment of the method described herein.

The biosensor 300 has a first photodiode 301 and a second photodiode 302 which are arranged in an insulator layer 303 made of insulator material such as silicon. The biosensor 300 furthermore has an oxide layer 304 and a second layer 305 located thereupon. The second layer 305 consists of a metal which is not suitable for immobilization of macromolecular biopolymers. The layer 305 may be composed of platinum, for example. On the layer 305 the units for immobilizing macromolecular biopolymers, which have the form of nanoparticles, are produced by the following method.

A solution of 0.5% by weight block copolymer polystyrene (PS)-block-poly(2-vinylpyridine) (P2VP) of the general formula PS(x)-b-P2VP(y) is admixed, as described in [12] and [13], with 0.5 equivalents of HAuCl₄.H₂O per pyridine unit to form monodisperse (micellarly dissolved) gold particles. In the formula, x and y indicate the number of base units according to the ratio between monomer and initiator.

After the formation of homogeneous micelles, a monolayer of nanoparticles made of gold is precipitated from this solution on the layer 305 by reduction with hydrazine, as described in [12] and [13]. Subsequently, the organic components of the precipitated micelles, i.e. the block copolymer, are removed from the layer 305 by plasma etching by means of an oxygen plasma (cf. [13]). The gold particles 306 which serve as the units for immobilizing macromolecular biopolymers remain intact during this treatment with plasma and form, as illustrated in the sectional view in FIG. 3b and the top view in FIG. 3c, a regular arrangement on the layer 305 (cf. [12]). The distances between the gold nanoparticles 306 are usually several 10 nm, e.g. approx. 20 to 30 nm. The size of the nanoparticles is preferably in the range from approx. 5 to 10 nm.

It is, of course, also possible to use, apart from the abovementioned block copolymers, other block copolymers for forming the nanoparticles.

Alternatively, the units 306 for immobilizing in the form of nanoparticles may be generated on the biosensor, as described in [14], by first forming a mask for the generation of nanostructures from colloidal particles on the layer 305 and then depositing gold particles for example by means of vacuum deposition.

After applying the nanoparticles 306 made of gold, the sensor 300 is structurized in such a way that the layer 305 made of platinum including the units 306 for immobilizing remains only on those regions which are located on the photodiodes 301, 302, as the sectional view in FIG. 3d and the top view in FIG. 3e indicate. This structurization is possible, for example, with the aid of any suitable familiar chemical etching method.

With the aid of the biosensor 300 designed in this way, it is possible to carry out the method for detecting macromolecular biopolymers, described in the first exemplary embodiment. FIG. 3f depicts a DNA scavenger molecule 307 immobilized on a gold nanoparticle 306 by means of gold-sulfur coupling.

The use of the biosensor 300 offers the advantage of the units 305 for immobilizing, present in the form of nanoparticles, making it possible to immobilize a precisely defined number of scavenger molecules. Therefore, preference is given to using the biosensor 300 for a quantitative detection of macromolecular biopolymers.

FIG. 6 depicts a biosensor 600 which can be used to carry out a redox recycling process according to another exemplary embodiment of the method of the invention.

The biosensor 600 has three electrodes, a first electrode 601, a second electrode 602 and a third electrode 603.

The electrodes 601, 602, 603 are electrically insulated from one another by means of an insulator material as insulator layer 604.

A holding region 605 for holding probe molecules capable of binding macromolecular biopolymers is provided on the first electrode 601. Said holding region may be configured as a uniform unit for immobilizing, but it is also possible to design said holding region with units for immobilizing in the form of nanoparticles.

The probe molecules (scavenger molecules) 606 according to this exemplary embodiment, which are immobilized on the holding region, are DNA probe molecules to which DNA strands having a sequence complementary to the sequence of said DNA probe molecules can hybridize. The probe molecules 606 carry at their 5′ terminus a biotin group as label 607, which may be attached there, for example, by using the “FluoReporter Biotin-X-C5 Oligonucleotide Labeling Kit” (Product No. F-6095) from Molecular Probes, Eugene, Oreg., USA (cf. 11).

The DNA probe molecules 606 are immobilized on the first electrode 601 made of gold by means of the known gold-sulfur coupling. When using a different material for binding the probe molecules, the material is provided with the appropriate coating material on which said probe molecules can be immobilized.

During immobilization of the DNA probe molecules on the first electrode 601, different electric potentials are applied to the electrodes so that an electric field between the electrodes is produced in such a way that immobilization of the DNA probe molecules is possible only at the first electrode 601 and is prevented at the second electrode 602 and/or at the third electrode 603.

Similarly to the method according to the prior art, as has been described above (cf. FIG. 4), in a further step a solution 609 to be studied, for example an electrolyte, which contains the macromolecular biopolymers possibly to be detected, i.e. the DNA strands which can hybridize with the DNA probe molecules, is contacted with the biosensor 600, i.e. in particular with the first electrode 601 and the labeled DNA probe molecules 606 located thereon. This is carried out in such a way that DNA strands 608 which may be present in the solution to be studied can hybridize with the DNA probe molecules 606.

Subsequently, scavenger molecules 606 to which no DNA strands to be detected have been hybridized are removed. This may be carried out by adding the DNA nucleases mentioned in the first exemplary embodiment above to the electrolyte 606. In this case, too, any of the following substances may be used for hydrolyzing the single-stranded DNA probe molecules 606:

nuclease from mung beans,

nuclease P1,

nuclease S1, or

DNA polymerases capable of breaking down single-stranded DNA, due to their 5′→3′ exonuclease activity or their 3′→5′ exonuclease activity.

After nuclease treatment, only the hybrids of labeled scavenger molecules 606 and DNA molecules 608 to be detected are present on the biosensor. This stage is depicted in FIG. 6a.

At this stage, the biosensor 600 is rinsed by means of a rinsing solution (not shown), i.e. the fragments of the nonhybridized DNA strands and the solution to be studied are removed.

In a next step, another solution (not shown) is contacted with the biosensor 600, in particular with the first electrode 601.

Said other solution contains an enzyme 610 which binds to the label 607 of the hybridized DNA probe molecules 606 and which can cleave the molecules illustrated below which are added in another solution 611.

Examples of enzymes 610 which may be used according to this exemplary embodiment are

alpha-galactosidase,

beta-galactosidase,

beta-glucosidase,

alpha-mannosidase,

alkaline phosphatase,

acidic phosphatase,

oligosaccharide dehydrogenase,

glucose dehydrogenase,

laccase,

tyrosinase,

or related enzymes.

In the present invention, the enzyme 610 is used in the form of an avidine conjugate. The reason for this is that avidine forms a specific bond with the biotin label 607 used herein by way of example (FIG. 6b).

It should be noted here that low molecular weight enzymes are able to ensure the highest conversion efficiency and therefore also the highest sensitivity when used as enzyme which effects the redox recycling.

The other solution 611 contains molecules 612 which can be cleaved by the enzyme 610 into a first part molecule 613 having a negative electric charge and into a second part molecule having a positive electric charge (cf. FIG. 6b).

Examples of the cleavable molecule 612 which may be used are above all:

p-aminophenyl hexylpyranoside,

p-aminophenyl phosphates,

p-nitrophenyl hexopyranosides,

p-nitrophenyl phosphates, or

suitable derivatives of diamines, catecholamines, Fe(CN)₆⁴⁻, ferrocene, dicarboxylic acid, ferrocenelysine osmium bipyridyl-NH, or PEG-ferrocene 2.

In this embodiment an electric potential is then applied in each case to the electrodes 601, 602, 603.

A first electric potential V(E1) is applied to the first electrode 601, a second electric potential V(E2) is applied to the second electrode 602 and a third electric potential V(E3) is applied to the third electrode 603.

During the actual measuring phase which takes place in principle in a manner similar to the procedure of the prior art, as has been described above, a following potential gradient of the electric potentials is applied, depending on the sign of the charge, in each case to the electrodes 601, 602, 603 in such a way that:

V(E3)>V(E1)>V(E2).

If, for example, the third electrode 603 has a positive electric potential V(E3), then the third electrode 603 has the largest electric potential of the electrodes 601, 602, 603 of the biosensor 600.

This causes the first part molecules 613 with negative charge generated to be attracted to the positively charged third electrode 603 and no longer, as in accordance with the prior art, to the first electrode 601, due to the largest electric potential V(E3) which is applied to the third electrode 603.

Consequently, the first electrode 601 is no longer used in this exemplary embodiment both as holding electrode for holding the probe molecules and as measuring electrode for oxidizing or reducing the particular part molecules. Rather the electrode 601 serves only to immobilize the probe molecules or the complexes of probe molecules and macromolecular biopolymers to be detected.

The third electrode 603 now takes over the function of the electrode at which oxidation or reduction of the part molecules generated takes place.

This means, by way of illustration, that the first electrode 601 is shielded from the cleaved part molecules by means of the third electrode 603.

In this way, and as another advantage of this embodiment, covering of the first electrode with the DNA probe molecules 606 can be increased considerably.

The negatively charged part molecules 613 are oxidized at the third electrode 603 and the oxidized first part molecules 614 are attracted to the second electrode 602, since the latter has the smallest electric potential V(E2) of all electrodes 601, 602, 603 in the biosensor 600.

The oxidized part molecules are reduced at the second electrode 602 and the reduced part molecules 615 are, in turn, attracted to the third electrode 603 where they are again oxidized.

In this way the present invention, similarly to the manner known in the prior art, results in a circuit current which is detected likewise in the known manner. Thus the resulting signal here is also a time course of the circuit current. From this it is in turn possible (by means of the enzyme 610 bound via the label 607) to calculate the number of the hybridized DNA strands 606 and thus of the DNA molecules 608 to be detected, owing to the proportionality of the circuit current to the number of charge carriers generated by the enzyme 610.

However, it should be noted here that this redox recycling method in accordance with the invention may also be carried out using the known “2-electrode arrangement” according to FIG. 4. In this case, however, it is not possible to utilize the advantage of the biosensor 600 described herein, which, due to the configuration of the electrode 601 as immobilization electrode, allows a higher covering density than the arrangement known from the prior art.

1. A method for detecting macromolecular biopolymers by using at least one unit for immobilizing macromolecular biopolymers, which method comprises
providing the at least one unit for immobilizing macromolecular biopolymers with scavenger molecules, it being possible for said scavenger molecules to bind macromolecular biopolymers and said scavenger molecules having a label which can generate a detectable signal,
contacting a sample to be studied with the at least one unit for immobilizing macromolecular biopolymers, it being possible for said sample to be studied to contain the macromolecular biopolymers to be detected,
binding macromolecular biopolymers present in the sample to be studied to the scavenger molecules,
removing scavenger molecules to which no macromolecular biopolymers to be detected have bound,
detecting the macromolecular biopolymers by using the label.

2. The method as claimed in claim 1, in which the label generates a signal.

3. The method as claimed in claim 2, in which the label is selected from the group consisting of fluorescent and chemiluminescent dyes, radioisotopes, enzymes and enzyme ligands.

4. The method as claimed in any of claims 1 to 3, in which the macromolecular biopolymers detected are nucleic acids, oligonucleotides, proteins or complexes of nucleic acids and proteins.

5. The method as claimed in claim 4,
in which the macromolecular biopolymers detected are proteins or peptides, and
in which the scavenger molecules used are ligands which can specifically bind the proteins or peptides.

6. The method as claimed in claim 5, in which unbound ligands are removed from the at least one unit for immobilizing by contacting a material with the at least one unit for immobilizing, said material being capable of hydrolyzing the chemical bond between the ligand and the unit for immobilizing.

7. The method as claimed in claim 6, in which the material being contacted with the at least one unit for immobilizing is an enzyme.

8. The method as claimed in claim 7, in which the enzyme being contacted with the at least one unit for immobilizing is a carboxylic ester hydrolase (esterase).

9. The method as claimed in claim 4, in which the macromolecular biopolymers detected are DNA or RNA molecules.

10. The method as claimed in claim 9,
in which the macromolecular biopolymers detected are DNA single strands having a predetermined nucleotide sequence, and
in which the scavenger molecules used are DNA probe molecules having a nucleotide sequence complementary to the predetermined nucleotide sequence.

11. The method as claimed in claim 10, in which unbound DNA probe molecules are removed from the at least one unit for immobilizing by contacting an enzyme having nuclease activity with the unit for immobilizing.

12. The method as claimed in claim 11, in which the enzyme having nuclease activity used is at least one of the following substances:
nuclease from mung beans,
nuclease P1,
nuclease S1, or
DNA polymerases capable of breaking down single-stranded DNA due to their 5′→3′ exonuclease activity or their 3′→5′ exonuclease activity.

13. The method as claimed in any of the preceding claims, in which the at least one unit for immobilizing is applied to an electrode or to a photodiode.

14. The method as claimed in any of the preceding claims, in which the unit for immobilizing is an arrangement of nanoparticles.
